(19) United States
(12) Patent Application Publication
(10) Pub. No.: US 2011/0106053 A1
(43) Pub. Date: May 5, 2011

(54) TREATMENT APPARATUS

(76) Inventor: Wayne Frederick Leech, Auckland (NZ)

(21) Appl. No.: 12/673,573

(22) PCT Filed: Aug. 14, 2008
(86) PCT No.: PCT/NZ08/00210

§ 371 Date: Apr. 26, 2010

(30) Foreign Application Priority Data
Aug. 14, 2007 (NZ) 560646

(51) Int. Cl. A61M 5/31

(52) U.S. Cl. 604/518; 604/87

(57) ABSTRACT

A treatment apparatus in the form of a bag which includes a first compartment containing at least one treatment substance, a second compartment containing at least one treatment substance, at least one openable seal between the first and second compartments, wherein at least one treatment substance in the first compartment is incompatible over time with at least one treatment substance in the second compartment.

### TECHNICAL FIELD

This invention relates to a treatment apparatus.

More specifically, this invention relates to a treatment apparatus for use in the treatment of a disorder in an animal.

### BACKGROUND ART

The treatment of medical disorders in animals often requires the administration of more than one treatment substance simultaneously, or in combination.

Two (or more) treatment substances may be required to treat a single condition of the animal. In other cases, two treatment substances for different conditions may be co-administered, thereby increasing efficiency. This is especially important when large numbers of animals are being treated.

A significant problem arises when the two (or more) treatment substances to be simultaneously administered are not compatible.

Incompatibility can arise from a number of causes, for example the substances may not be stable in admixture in that they may separate or react differently with the external environment or alternatively chemical reactions may occur between different substances or components in the mixture.

If the substances are incompatible they cannot be manufactured as a single product, nor can they be premixed and allowed to stand for any significant time period prior to administration.

This incompatibility also means that a premixed solution, if made, could not be stored without subsequent deactivation through some means prior to administration.

In cases where two or more incompatible substances need to be administered, a veterinarian, or any other person (such as a farmer) administering the solution has to mix the treatment substances together on site, immediately prior to administering same, or alternatively, simply administer the substances sequentially.

In addition to the problem of one or more incompatible treatment substances causing chemical or physical degradation of the admixture, the process of mixing substances immediately prior to administration can cause a number of other significant disadvantages.

One disadvantage is that in some cases, such as with the administration of metabolic supplements, additional time and effort is required to prepare the correct dose of the substances to be administered for each animal. This reduces the efficiency of administration.

When treating numerous animals, such as in a herd or flock, this inefficiency multiplies significantly.

Another disadvantage is that there is the risk of errors being made in preparation of doses. This risk may be augmented when such administration occurs under a restricted time constraint or other distractions.

If the treatment substances are incorrectly measured or mixed, this can in some cases have a serious health impact on the animal. For example, they may be given a dose which is so low that it is ineffective; alternatively they may be given a dose which is so high that it is toxic.

Similarly it may be that the ratios of the substances to each other may be critical so any variation between these may lead to efficacy or toxicity issues.

A further disadvantage of mixing incompatible treatment substances immediately prior to administration is that there is an increased risk of introduction of contaminants to the dosage mixture, for example, when treatment substances are mixed in an environment which is neither sterile nor controlled, for example a farm shed, yard or paddock. Many of these environments may contain very high levels of contaminants, especially if animals in a diseased state or otherwise in low health are present.

It will be appreciated by the skilled reader that in addition to the risk of introduction of microbial contaminants, some treatment substances may physically react with chemical contaminants present in the environment where mixing occurs. The presence of water can greatly influence the physical state of hygroscopic substances, sometimes to the extent that they become impossible to handle. Similarly, substances which react with oxygen may degrade if exposed to the air.

Not only can contamination during the mixing process result in detrimental contaminants gaining access to the treatment substances before being administered to any given animal, but also, if the mixture is left in bulk vessel from which subsequent doses are drawn over a long period of time such contamination may languish in the mixture causing increasing detriment. Alternatively, such a vessel may be at risk of further contamination and degradation. This is highly undesirable.

In particular, the contaminants may as a consequence of the treatment process (e.g. intravenous injection) be introduced directly into the animal's system, bypassing normal defence mechanisms (that is, those which would occur if the dosage entered the system via the gastrointestinal tract). In these situations the treatment process may-cause more harm than good.

One way to overcome the problems of on-site mixed dosage preparation is to pre-package the different treatment substances in separate containers of predetermined size, which can then be readily mixed together. The significant problem with this method is that the dosage rate may vary widely, for example based on animal weight, the animal type, or the degree of sickness or treatment required.

In these situations the dosage required would be based on the total volume of the mixed treatment substances (at a specific ratio to one another). Therefore, again in this case, if the dosage is not a constant and unvarying amount the veterinarian or person administering the dosage will again have to measure out each substance and mix these together.

Alternatively the substances could be mixed together in a bulk vessel and the desired volume withdrawn for individual dosing. However, this requires the substances to be properly and evenly mixed. Obviously this may not always happen in the field, with undesirable consequences as outlined above.

One of the major problems associated with the above methods is that it can be difficult to handle unstable treatment substances on site. They may require specialist treatment to ensure safe handling. Again this decreases the ease with which the treatment substances can be measured, mixed and administered on site.

A further alternative to mixing treatment substances on site prior to administration is to administer the treatment substances to the animal sequentially. While this overcomes problems associated with contamination and measuring out substances, this can lead to additional trauma to the animal, especially if the substances are being injected. In addition, it is time inefficient and physically cumbersome for the veterinarian or other person to have to dose the same animal twice or more.

It would therefore be desirable to have an apparatus and/or method for delivering one or more substances to an animal in a way which simultaneously overcomes the issues of improper mixing, contamination, and inefficient multiple dosing as discussed above.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a treatment apparatus in the form of a bag including,

a first compartment containing at least one treatment substance,

a second compartment containing at least one treatment substance,

at least one openable seal between the first and second compartments,

characterised in that at least one treatment substance in the first compartment is incompatible over time with at least one treatment substance in the second compartment.

According to another aspect of the present invention there is provided a method of administering at least two substantially incompatible treatment substances to an animal, using a treatment apparatus in the, form of a bag including:

a first compartment containing at least one treatment substance,

a second compartment containing at least one treatment substance,

at least one breakable seal between the first and second compartments,

wherein at least one treatment substance in the first compartment is incompatible over time with at least one treatment substance in the second compartment,

the method characterised by the steps of:
a) opening the openable seal(s) between the first and second compartments of the treatment apparatus,
b) mixing the incompatible treatment substances from the first and second compartments,
c) administering the mixed treatment substances to the animal.

Throughout this specification the term “openable” should be taken as meaning broken, removed, or opened in such a away that material can pass through it from one compartment to another.

In one preferred embodiment the treatment apparatus, herein referred to as a bag may have two compartments.

However, this should not be seen as limiting, the bag may include more than two compartments if more than two treatment substances are to be delivered. For example if three treatment substances are to be delivered, the bag may contain three separate compartments.

One skilled in the art would realise that if the bag had more than two compartments it could also include more than one breakable seal. For example if the bag had three compartments one beside the other in a row, then there may be at least one breakable seal between the first and second compartments, and at least one breakable seal between the second and third compartments.

Alternatively the compartments may be arranged such that they have a common point or boundary, for example at an apex. In this example, one breakable seal, when broken may allow the treatment substances from all compartments to be administered at once.

Throughout this specification the term ‘compartment’ should be taken as meaning a portion or part of the bag which is separated from other portions or parts of the bag such that the treatment substances in adjacent compartments are prevented from mixing or coming into contact with one another until administration.

It should be appreciated that the size and shapes of the compartments may vary considerably. For example, in the situation where two incompatible treatment substances are to be delivered to an animal, the size and shape of the compartments may be designed and optimised based on the relative volume of each treatment substance required. For example, if the two treatment substances were required in approximately equal volumes, the bag may be divided into two compartments which are substantially the same size. Alternatively, if a much larger volume of one substance is required the bag may be divided into one large and one small compartment.

In one preferred embodiment, where the dosage of the treatment substances depends on animal size/weight, the bag may come in a range of pre-packed sizes, suitable for a particular animal size/weight range.

In a preferred embodiment at least two compartments of the bag may be joined via a seal, which blocks a conduit between the compartments, until broken or otherwise removed just prior to administration.

The seal may be positioned in such a way that when open, it would provide an inter-compartmental conduit through which treatment substances can flow.

In one preferred embodiment, the seal includes a short tube.

The tube may preferably be blocked at one end, thus completing a seal to separate the two compartments.

In a preferred embodiment, the blocked end of the tube is distal to the compartment from which it is intended the medicament should flow. The tube and seal may be a single unit constructed of a frangible material enabling the tube to be manually snapped or broken, thus breaking the seal.

In an alternative embodiment, the tube may be blocked at one end with a removable stop, such as a bung, plug or cap.

One skilled in the art will also appreciate that there are other methods of providing a conduit and seal between compartments without departing from the substance of the invention.

In a preferred embodiment the mixture of incompatible solutions may be designed for intravenous administration, and shall be referred to as such herein.

However, this should not be seen as limiting as other injection routes may also be utilised, such as intramuscular. Non-injection routes may also be utilised, for example; oral or external administration (for example a pour-on or drench so that the mixture can be absorbed through the skin).

In a preferred embodiment the bag may be manufactured from any deformable material.

In one preferred embodiment the material is polyvinyl chloride. However, this should not be seen as limiting as one skilled in the art will appreciate that there are a number of ways to provide an inter-compartmental membrane or barrier from a given suitable material including but not limited to heat welding a PVC bag at a given place or places to form two or more compartments. The one or more inter-compartmental conduits may be incorporated into the bag during this process. In an alternative embodiment the two or more compartments in the bag may be formed by arranging one or more smaller bags within the body of a larger bag.

Throughout this specification the term ‘treatment substance’ should be taken as meaning any compound, solution or mixture of compounds or solutions which provide a benefit to the animal, or are used to treat a condition (disease state).

It should be appreciated that one treatment substance may be used to treat a condition, while the other treatment substance allows for administration, for example a carrier, or provides the desired characteristics of the final mixture required or desired for administration.

In some embodiments the two treatment substances may both be in solution. However, this should not be seen as limiting, as in some instances one or, more of the treatment substances may be in the form of a liquid suspension, or alternatively in a dried, powdered or other solid form. This may especially be the case for substances which are not stable in solution. It should be appreciated that in these cases, the bag may contain one powdered treatment substance in one compartment, and a liquid solution in the other, into which the powder is dissolved or mixed just prior to administration.

Treatment substances used in the present invention may vary widely, and be for widely different purposes. For example treatment substances may include: vaccines, medications, vitamins or trace minerals.

Throughout this specification the term ‘incompatible’ should be taken as meaning at least two substances which are not capable of being stored together. This may be due to chemical reactions between the two substances, breakdown of at least one substance, or any other reaction or physical change incurred during storage of the two substances together which leads to inactivation of, or inefficient subsequent action of at least one of the treatment substances.

Alternatively, it may be that the bag is used to hold and deliver at least one treatment substance which is not stable in the physical form in which it is to be administered. For example a given substance may be unstable in solution, but due to efficacy issues the desired vehicle for administration may be a liquid—for example where a substance has to be administered by injection. In this case, the bag may have one compartment which contains a powdered form of a treatment substance and another compartment which contains a liquid carrier. At the point of administration the inter-compartmental seal can be broken and the two substances can be mixed and delivered.

It will be appreciated by the skilled reader that the bag may also be used to provide a suitable delivery mechanism for substances which are sensitive to air or water.

The term ‘incompatible’ does not in the present invention relate to treatment substances which should not be taken or administered simultaneously.

In one particularly preferred embodiment of the present invention one of the treatment substances may be Vitamin B₁₂.

Throughout this specification the term ‘Vitamin B₁₂’ should be taken as including any physiologically effective equivalent of Vitamin B₁₂ which is incompatible with other substances being used. These may include cobalamines and cyanocobalamins, or any determinable equivalents of Vitamin B₁₂ by reference to definition of Vitamin B₁₂ in its various guises in The Merck Index.

In one preferred embodiment the concentration of Vitamin B₁₂ may be approximately 0.05 to 0.5% (w/v) prior to mixing, for example 1000 μL/ml in Prolaject 1000. In this situation the concentration of Vitamin B₁₂ after mixing would be 0.001 to 0.003% (w/v).

One skilled in the art would realise that the concentration of Vitamin B₁₂ provided above is not limiting and could vary depending on the use, the size and type of the animal, the treatment to be administered, and the severity of the condition to be treated.

Similarly, the concentration of Vitamin B₁₂ after mixing will also vary depending on the volume of Vitamin B₁₂ when compared to the volume of the at least one other incompatible substance.

Vitamin B₁₂ is an important requirement for most animals. Vitamin B₁₂ is a cobalt-containing vitamin required by cells throughout the body for conversion of ribose nucleotides into deoxyribose nucleotides, a major step in the formation of deoxyribonucleic acid (DNA). Thus it is an essential nutrient for nuclear maturation and cell division. A deficiency in vitamin B₁₂ results in a form of anaemia. However, anaemias from deficiencies of vitamin B₁₂ are less apparent clinically in domestic animals compared with humans.

For example adult ruminants are not dependent on a dietary source of vitamin B₁₂ because bacteria within the rumen synthesise all the supplies of vitamin B₁₂ needed. However, cobalt is required by the ruminal micro-organisms to synthesise this vitamin B₁₂, and a deficiency in cobalt can, therefore, cause a deficiency of the vitamin.

In a preferred embodiment the vitamin B₁₂ formulation may have a pH in the range of substantially 5 to 7 prior to mixing with at least one other incompatible substance.

In a preferred form, the vitamin B₁₂ formulation may have a pH in the range of substantially 3.0 to 4.0, and more preferably 3.3 to 3.8 after mixing with at least one other incompatible substance.

One skilled in the art would realise that these pH values should not be limiting. The pH (either before or after mixing) will also depend on the condition to be treated and the substances which are to be mixed, this is to ensure that all substances are in an active and effective form on administration.

It is well known that vitamins are hygroscopic and relatively more stable in a dry (pure crystal form).

Therefore vitamin B₁₂ could be present in a compartment in a crystal form. In this embodiment it would be preferable for the crystalline vitamin B₁₂ to be positioned in the lower compartment (when the bag is in the correct position for mixing and administration). This would allow a liquid from an upper compartment to flow into the compartment containing the vitamin B₁₂ and act as a carrier for the vitamin B₁₂ during administration. It should be appreciated that preferably the liquid is one in which vitamin B₁₂ is substantially soluble. However, this should not be seen as limiting, as in an alternative embodiment the liquid may simple pick up and carry the crystalline vitamin B₁₂ during administration. In this case, it is important to ensure that the correct dose can be administered.

In a preferred embodiment the second treatment substance when delivered with Vitamin B₁₂, may be glucose:

One skilled in the art would readily realise that all Vitamin B₁₂ forms are incompatible with reducing sugars, such as glucose due to the Maillard reaction. This is due to a reaction between the amines and amides in Vitamin B₁₂ reacting with the reducing sugar. This undesirable reaction occurs for all modified forms of vitamin B₁₂ usually within six hours of mixing. Therefore, these substances can only be mixed within six hours of administration. This reaction is not dependent on chemical conditions, such as pH.

In a preferred embodiment the concentration of glucose may be substantially 25% in solution, such as water. As glucose has a high solubility, high concentrations can be utilised with the present invention. These are also easy for the animal being treated to absorb and metabolise.

Glucose is a preferred sugar for use with the present invention as it is easily metabolised by the animal. However the use of glucose should not be seen as limiting, as any other reducing sugar could also be utilised with the present invention.

Alternatively the treatment substance may be any other compound, which is incompatible with Vitamin B₁₂, but provides benefit to the animal or acts to treat a metabolic condition or disorder. Examples include, but are not limited to other sugars, phosphorous, calcium, magnesium or any other mineral, trace element or vitamin such as selenium or amino acids.

In one preferred embodiment, at least one of the treatment substances may include at least one other treatment substance or pharmaceutically acceptable compound.

For example, in the situation described above where the bag is used to administer vitamin B₁₂ and glucose, one of these compartments may also include calcium. In this case, calcium is compatible with both substances, so could be incorporated into either compartment.

It should be appreciated that more than one additional substance may be included with one or both of the incompatible treatment substances.

In one preferred embodiment, calcium may be included in the glucose mixture. This would allow the utilisation of an existing product of the applicant's, Glucalmax®. Glucalmax® is a combination of calcium borogluconate, dextrose and magnesium chloride, in solution for the treatment of milk fever.

In one embodiment the bag of the present invention may contain the combinations as set out in example 3, 4 and 5 (2 and 3 of Best Modes Section), being:

| Example | Compartment 1 | Compartment 2 |
|---|---|---|
| 3 | 20 mL Prophos ® injection | 500 mL Glucalmax ® |
| 4 | 10 mL modified Prolaject ® B₁₂ | 500 mL Glucalmax ® |
| 5 | 10 mL modified Prolaject ® B₁₂ | 500 mL Glucalphos ® |

However, these are given as examples only, and one skilled in the art would readily realise that a wide range of incompatible components and combination thereof may be administered by the method and apparatus of the present invention.

Similarly, depending on the desired treatment and species of animal to be treated, the volumes or ratio thereof may differ from those examples provided above.

In one embodiment the recommended dosages are 500 ml (one bag) for cattle injected by slow intravenous route for the treatment of parturient paresis (milk fever) and milk fever complicated by acetonaemia and hypomagnesaemia (grass staggers) and as an aid in restoring appetite and blood glucose levels in cattle deficient in cobalt. The dosage may be repeated after 4-6 hours if necessary. The volume administered each time may vary significantly dependant on the state of the animal. This would be judged by the vet or farmer at the time.

In Example 5, in addition to the dosage above for cattle, will also include a dosage for sheep of 100 ml by subcutaneous route in the neck or over the ribs (or 100 ml intravenously). This is for the treatment of milk fever in sheep and as an aid in restoring normal appetite and blood glucose level in sheep deficient in cobalt.

In one embodiment the treatment for sheep may still be available sold in the same bag and same volumes but with only 100 ml being administered at a time.

In this instance in preferred embodiments the bag may include an approximate scale on the side of the bag to aid in determining the correct volume to administer.

In an alternative embodiment smaller Bags may be utilised of which are configured to hold the correct volume (e.g. 100 mL) for each administration to sheep.

It should be appreciated that the seal may be broken at any time prior to administration provided that the mixture does not languish for a period longer than that in which the two or more treatment substances are temporarily stable. For example Vitamin B₁₂ and glucose are temporarily stable when mixed for up to six hours, therefore the seal may be broken, and these substances mixed up to six hours prior to administration. This may be advantageous, for example when a large number of animals are to be treated; the seals may be broken on all bags, prior to starting to treat the herd. This may be done while animals are being herded, moved into the yard, and so on.

In a preferred embodiment the bag of the present invention may also include at least one outlet. Once the incompatible treatment substances have been mixed, it is necessary to be able to quickly and easily administer the mixed substance to the animal without having to cut open the bag, or inset needles or other apparatus to the bag.

In a preferred embodiment, the outlet may be in the form of a tube which can be connected to a needle, for injection into the animal. However, this should not be seen as limiting as the outlet may also be in other forms, for example a larger outlet which allows the mixed solution to be administered as a drench or pour on.

In some embodiments the bag may also include at least one inlet to each compartment. This allows the compartments to be quickly and easily filled.

In a preferred embodiment the inlet may be in the form of a tube. It should be appreciated that the inlet should be air tight once the compartment has been filed, this will prevent leakage, either of the substance out of the bag, or other components into the bag, for example air or water.

In one embodiment the inlet may include a stopper which allows an additional substance to be introduced prior to administration. This may be for example via a stopper with a rubber septum which allows a substance to be injected into the compartment through a needle or syringe while retaining an airtight seal.

The bag and method of the present invention provide a significant number of advantages. These include the following:
The bag and method allow a one step administration to an animal of at least two treatment substances which are incompatible and/or are unable to be pre-mixed and stored together.
The multiple compartment bag with one or more breakable seals between the compartments provides a quick and easy means of mixing the correct dosages of different substances together on site for administration to an animal.
The incompatible treatment substances are maintained in separate compartments in a stable state until just prior to administration.
The sealed, pre-measured dose contained in the bag do not suffer from problems associated with the user errors in measuring and mixing the treatment substances on site in a non-sterile environment.
The bag and method of the present invention provides a time and labour efficient method of administering treatment to animals. This is highly beneficial in situations where animals may be in distress and uncooperative.

### BRIEF DESCRIPTION OF DRAWINGS

Further aspects of the present invention will become apparent from the following description which is given by way of example only and with reference to the accompanying drawings in which:

FIG. 1 shows a schematic of a bag according to one preferred embodiment of the present invention,

### BEST MODES FOR CARRYING OUT THE INVENTION

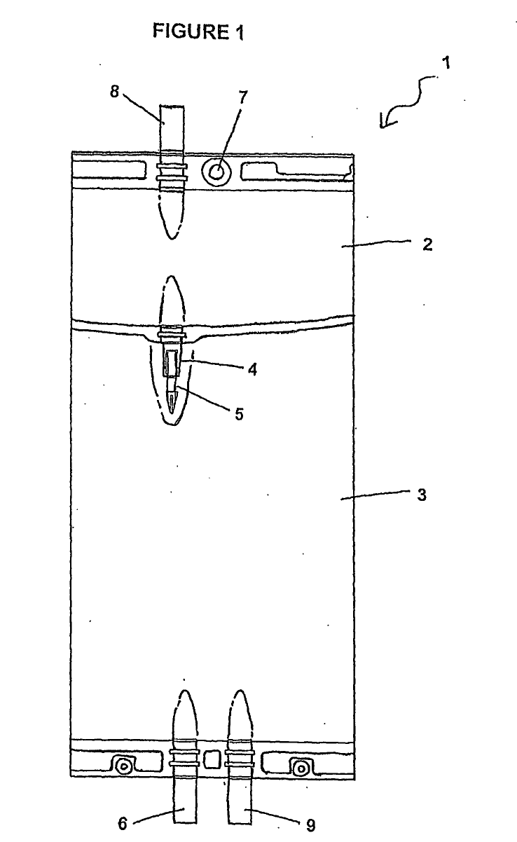

FIG. 1 shows a bag (1) according to one aspect of the present invention.

The bag (1) has two compartments (2) and (3). Each compartment is completely sealed off from the other, allowing bag (1) to hold two (or more) incompatible treatment substances, one in each compartment (2) and (3).

The two compartments (2 and 3) are joined by a seal (4). The seal is a ‘plastic plug’ similar to those found on the ends of tubes, for example of glue. Once the end of the seal (5) is broken off, a conduit between the two compartments (2) and (3) is open, and the treatment substances in the two compartments can mix.

The bag also includes an outlet (6) from which the mixed contents of the compartments (2 and 3) can be administered to an animal.

As shown in FIG. 1 the bag in use would be positioned such that it is held up by a reinforced aperture (7). In this case, compartment (2) would be the upper compartment and compartment (3) would be the lower compartment. Once mixed the treatment substances can be gravity feed through the outlet to the animal via a tube and needle (not shown).

The bag (1) also includes inlets (8 and 9) to each compartment (2 and 3) respectively. These allow the compartments to be filled with suitable treatment substances.

### EXAMPLES

Examples are now provided showing various embodiments of the present invention.

### Example 1

Example 1 sets out the loss of efficiency of incompatible treatment substances when mixed (i.e. when the treatment apparatus of the present invention is not utilized).

The stability of the following solutions (based on the concentration of vitamin B₁₂ in the form of cyanocobalamin) was analyzed over time:

Solution1:

Cyanocobalamin (0.006% w/v) was added to a metabolic solution containing dextrose (22% w/v), calcium borogluconate (18% w/v), magnesium chloride (3% w/v) and water.

Solution 2:

Cyanocobalamin (0.0035% w/v) was added to a second sample of metabolic solution containing dextrose, (22% w/v), calcium borogluconate (18% w/v) magnesium chloride (3% w/v), magnesium hypophosphite (5% w/v) and water.

The solutions were stored at various temperatures in sealed vials and the content of cyanocobalamin tested by HPLC analysis at various time points.

Results

The results of the trial for solution 1 are presented in Table 1.

TABLE 1

| | Concentation of | Percentage of initial |
|---|---|---|
| Storage Conditions | Cyanocobalamin (ppm) | concentration (%) |
| 3 days/55° C. | 64.2 | 98.6 |
| 3 days/85° C. | 38.8 | 59.6 |
| 1 month/25° C. | 64.2 | 98.6 |
| 1 month/55° C. | 46.3 | 71.1 |

The results of the trial for solution 2 (containing magnesium hypophosphite) are presented in Table 2

TABLE 2

| | Concentration of | Percentage of initial |
|---|---|---|
| Storage Conditions | Cyanocobalamin (ppm) | concentration (%) |
| 1 day/55° C. | 17.0 | 65.9 |
| 1 day/85° C. | 0.12 | 0.5 |

Both solutions show chemical instability for cyanocobalamin content over a period of time. The cycanocobalamine concentration in Solution 1 dropped to 71.1% of initial levels over 1 month at 55° C. Solution 2 which included magnesium hypophosphite, dropped in concentration of cyanocobalamin to 65.9% after only one day under the same conditions.

These solutions did not display a sufficient shelf life to be commercially viable.

### Example 2

In a second trial a two compartment bag of the present invention was utilized to hold four known nutritional and metabolic solutions in various combinations. The nutritional and metabolic solutions were as follows:
1. Glucalmax injection: containing Calcium borogluconate 25% w/v, Dextrose and Magnesium chloride 4% w/v.
2. Glucalphos injection: containing Calcium borogluconate 25%w/v, Dextrose and Magnesium hypophosphite 5% w/v.
3. Prolaject B₁₂ 1000 injection: containing 0.1% w/v of Hydroxocobalamin.
4. Prophos injection: containing 12.5% w/v of sodium oxybenzylphosphinic acid, 0.9% w/v sodium dihydrogen phosphonate and 0.005% w/v of Cyanocobalamin.

The combinations trialed are shown in Table 3.

TABLE 3

| | Combination 1 | Combination 2 | Combination 3 |
|---|---|---|---|
| Upper | 10 ml Prolaject B₁₂ | 10 ml Prolaject B₁₂ | 20 ml Prophos injection |
| Compartment | | | |
| Lower | 500 ml Glucalmax | 500 ml Glucalphos | 500 ml Glucalmax |
| Compartment | | | |

The seal was broken between compartments and the solutions mixed at room temperature. Hydroxocobalamin (or in combination 3, cyanocobalamin) content in the mixture was assayed by HPLC analysis at time intervals up to 6 hours after mixing. The solutions also underwent visual inspection for colour change at time intervals up to 24 hours after mixing.

Results

The results of the visual inspection at 2 hourly intervals over 24 hours are given in Table 4.

TABLE 4

| | Change in colour following mixing | | | | | | | | | | |
|---|---|---|---|---|---|---|---|---|---|---|---|
| | at various time points in hrs | | | | | | | | | | |
| | 0 | 2 | 4 | 6 | 12 | 14 | 16 | 18 | 20 | 22 | 24 |
| Combination 1 | No | No | No | No | No | No | No | No | No | No | No |
| Combination 2 | Yes | Yes | Yes | Yes | Yes | Yes | Yes | Yes | Yes | Yes | Yes |
| Combination 3 | No | No | No | No | No | No | No | No | No | No | No |

Where “No” represents no change in colour and Yes represents a change in colour.

The results of the HPLC analysis of levels of Hydroxocobalamin or Cyanocobalamin at time intervals over 6 hours are shown in Table 5.

TABLE 5

| | Time intervals (hours after mixing) | | | |
|---|---|---|---|---|
| | 0 | 2 | 4 | 6 |
| Combination 1 (content of | 0 | 0 | 0 | 0 |
| Hydroxocobalamin % w/v) | | | | |
| Combination 2 (content of | 0 | 0 | 0 | 0 |
| Hydroxocobalamin % w/v) | | | | |
| Combination 3 (content of | 0.00024 | 0.00024 | 0.00024 | 0.00024 |
| Cyanocobalamin % w/v) (required | | | | |
| content 0.00018-0.00033% w/v) | | | | |

The HPLC analysis of combination 1 and 2 showed a shift in the retention time of the peak for Hydroxocobalamin. The mixtures of Glucalmax and Prolaject B₁₂ or Glucalphos and Prolaject B₁₂ do not have chemical stability when mixed within the invention however, the mixture of Glucalmax and Prophos (in combination 3) did show chemical stability for cyanocobalamin over 6 hours.

### Example 3

In a further trial the inventors modified the Prolaject B₁₂ 1000 injection to contain 0.1% of Cyanocobalamin instead of Hydroxocobalamin. This modified Prolaject B₁₂ solution was put in the upper compartment of an embodiment of the invention containing two compartments and made of PVC. Glucalmax or Glucalphos were put in the lower compartment as detailed in Table 6.

TABLE 6

| | Combination 4 | Combination 5 |
|---|---|---|
| Upper | 10 ml modified | 10 ml modified |
| Compartment | Prolaject B₁₂ | Prolaject B₁₂ |
| Lower | 500 ml Glucalmax | 500 ml Glucalphos |
| Compartment | | |

The seal was broken between compartments and the solutions mixed at room temperature. Cyanocobalamin content in the mixture was assayed by HPLC analysis at time intervals up to 6 hours after mixing.

Results

The results of the HPLC analysis are given in Table 7

TABLE 7

| | Time points (hours after mixing) | | | |
|---|---|---|---|---|
| | 0 | 2 | 4 | 6 |
| Combination 4 (content of | 0.0025 | 0.0025 | 0.0024 | 0.0024 |
| Cyanocobalamin % w/v) (required | | | | |
| content 0.0018-0.0033% w/v) | | | | |
| Combination 5 (content of | 0.0025 | 0.0025 | 0.0024 | 0.0024 |
| Cyanocobalamin % w/v) (required | | | | |
| content 0.0018-0.0033% w/v) | | | | |

The content of cyanocobalamin stays within the required range by HPLC analysis for 6 hours when both combination 4 and combination 5 are mixed within the invention.

This makes it possible for these nutritional and metabolic solutions to be supplied and administered together with the invention.

Aspects of the present invention have been described by way of example only and it should be appreciated that modifications and additions may be made thereto without departing from the scope thereof as defined in the appended claims.

1.-40. (canceled)

41. A treatment apparatus in the form of a bag configured for connection to a needle for application of treatment substances to an animal,
wherein the bag comprises:
a first compartment containing at least one treatment substance,
a second compartment containing at least one treatment substance,
at least one openable seal between the first and second compartments,
wherein
at least one treatment substance in the first compartment is incompatible, within three days at a temperature of 85° C. or less, with at least one treatment substance in the second compartment, and
the configuration of the bag is such that, upon opening of the openable seal, the two incompatible treatment substances can mix with each other in a compartment, and subsequently exit through a single port for administration through a needle.

42. A treatment apparatus as claimed in claim 41, wherein the bag has at least three compartments.

43. A treatment apparatus as claimed in claim 41, wherein the bag is a pre-packed size suitable for a particular animal size/weight range.

44. A treatment apparatus as claimed in claim 41, wherein the bag includes more than one openable seal.

45. A treatment apparatus as claimed in claim 41, wherein the compartments are arranged such that they have a common point or boundary.

46. A treatment apparatus as claimed in claim 41, wherein the seal is constructed of frangible material.

47. A treatment apparatus as claimed in claim 41, wherein the seal is positioned in such a way that when open, it provides an inter-compartmental conduit through which treatment substances can flow.

48. A treatment apparatus as claimed in claim 41, wherein the seal includes a short tube.

49. A treatment apparatus as claimed in claim 48, wherein the tube is blocked at one end.

50. A treatment apparatus as claimed in claim 49, wherein the blocked end of the tube is distal to the compartment from which it is intended the treatment substance should flow.

51. A treatment apparatus as claimed in claim 48, wherein the tube is blocked at one end with a removable stop.

52. A treatment apparatus as claimed in claim 41, wherein the bag is manufactured from a deformable material.

53. A treatment apparatus as claimed in claim 41, wherein the bag is made substantially of polyvinyl chloride.

54. A treatment apparatus as claimed in claim 41, wherein one of the treatment substances is Vitamin B₁₂.

55. A treatment apparatus as claimed in claim 41, wherein the bag includes a scale on a side of the bag to aid in determining the correct volume to administer.

56. A treatment apparatus as claimed in claim 41, wherein the bag also includes at least one outlet.

57. A treatment apparatus as claimed in claim 56, wherein the outlet is in the form of a tube capable of connection to a needle for injection into the animal.

58. A treatment apparatus as claimed in claim 41, wherein the bag also includes at least one inlet to each compartment.

59. A treatment apparatus as claimed in claim 58, wherein the inlet includes a stopper.

60. A treatment apparatus as claimed in claim 58, wherein the inlet allows a substance to be injected into the compartment through a needle or syringe while retaining an airtight seal.

61. A method of administering at least two substantially incompatible treatment substances to a non-human animal, using a treatment apparatus in the form of a bag configured for connection to a needle for application to an animal, comprising:
a first compartment containing at least one treatment substance,
a second compartment containing at least one treatment substance,
at least one openable seal between the first and second compartments,
wherein at least one treatment substance in the first compartment is incompatible within three days at a temperature of 85° C. or less with at least one treatment substance in the second compartment,
the method including:
a) opening the openable seal(s) between the first and second compartments of the treatment apparatus,
b) mixing the incompatible treatment substances from the first and second compartments,
c) administering the mixed treatment substances through a single port for administration through a needle to the animal.

62. A method of administering a treatment substance as claimed in claim 61, wherein the two treatment substances are both in solution.

63. A method of administering a treatment substance as claimed in claim 61, wherein the bag is used to hold and deliver at least one treatment substance which is not stable in the physical form in which it is to be administered.

64. A method of administering a treatment substance as claimed in claim 61, wherein one of the treatment substances is Vitamin B₁₂.

65. A method of administering a treatment substance as claimed in claim 64, wherein the concentration of Vitamin B₁₂ is 0.05 to 0.5% (w/v) prior to mixing and the concentration of Vitamin B₁₂ after mixing would be 0.001 to 0.003% (w/v).

66. A method of administering a treatment substance as claimed in claim 64, wherein the vitamin B₁₂ formulation has a pH in the range of substantially 5 to 7 prior to mixing with at least one other incompatible substance.

67. A method of administering a treatment substance as claimed in claim 64, wherein the vitamin B₁₂ formulation has a pH in the range of substantially 3.0 to 4.0 after mixing with at least one incompatible substance.

68. A method of administering a treatment substance as claimed in claim 64, wherein the vitamin B₁₂ formulation is a pH in the range of 3.3 to 3.8 after mixing with at least one other incompatible substance.

69. A method of administering a treatment substance as claimed in claim 64, wherein vitamin B₁₂ is present in a compartment in a crystal form.

70. A method of administering a treatment substance as claimed in claim 69, wherein crystalline vitamin B₁₂ is positioned in a lower compartment of the bag when said bag is in the correct position for mixing and administration.

71. A method of administering a treatment substance as claimed in claim 61, wherein the second treatment substance or carrier is one in which vitamin B₁₂ is substantially soluble.

72. A method of administering a treatment substance as claimed in claim 61, wherein the second treatment substance or carrier is a reducing sugar.

73. A method of administering a treatment substance as claimed in claim 61, wherein the second treatment substance is glucose.

74. A method of administering a treatment substance as claimed in claim 73, wherein the concentration of glucose is substantially 25% (w/v) in solution.

75. A method of administering a treatment substance as claimed in claim 73, wherein calcium is included in the glucose mixture.

76. A method of administering a treatment substance as claimed in claim 61, wherein at least one treatment substance includes at least one other treatment substance or pharmaceutically acceptable compound.

77. A method of administering a treatment substance as claimed in claim 61, wherein the inlet includes a stopper.

78. A method of administering a treatment substance as claimed in claim 61, including the step of injecting a substance into the compartment through a needle or syringe while retaining an airtight seal.
